(19) United States
(12) Patent Application Publication
(10) Pub. No.: US 2017/0014365 A1
(43) Pub. Date: Jan. 19, 2017

(54) Compositions Containing Very Long Chain Saturated Fatty Acids and Methods of Use

(71) Applicant: The Board of Regents of the University of Oklahoma, Norman, OK (US)

(72) Inventors: Robert Eugene Anderson, Edmond, OK (US); Martin-Paul Agbaga, Oklahoma City, OK (US); Richard S. Brush, Midwest City, OK (US); Blake Hopiavuori, Oklahoma City, OK (US)

(21) Appl. No.: 15/210,469

(22) Filed: Jul. 14, 2016

Related U.S. Application Data
(60) Provisional application No. 62/192,381, filed on Jul. 14, 2015.

(51) Int. Cl. A61K 31/20; A23L 33/12

(52) U.S. Cl. CPC A61K 31/20; A23L 33/12; A23V 2002/00

(57) ABSTRACT

A fatty acid composition comprising at least one purified very long chain saturated fatty acid (VLC-SFA) having a chain length in a range of from about 28 carbons to about 40 carbons which is conjugated to a carrier molecule, forming a VLC-SFA-carrier conjugate. Nutritional supplements containing the fatty acid compositions, and methods of producing and using the fatty acid compositions, are also disclosed.

### CROSS REFERENCE TO RELATED APPLICATIONS/INCORPORATION BY REFERENCE STATEMENT

This application claims benefit under 35 USC §119(e) of Provisional Application U.S. Ser. No. 62/192,381, filed Jul. 14, 2015, the entirety of which is hereby expressly incorporated herein by reference.

### STATEMENT REGARDING FEDERALLY SPONSORED RESEARCH OR DEVELOPMENT

This invention was made with government support under Grant Number 1R21NS090117-01/02 and R01 EY04149-33/36 awarded by the National Institutes of Health. The government has certain rights in this invention.

### BACKGROUND

ELOngation of Very Long chain fatty acids-4 (ELOVL4) is an elongase responsible for biosynthesis of very long chain (≧C28) fatty acids, found as components of complex lipid molecules. ELOVL4 synthesizes the very long chain polyunsaturated fatty acids (VLC-PUFAs) in retina and testes, and very long chain saturated fatty acids (VLC-SFAs) in skin and brain. Case studies have reported that homozygous inheritance of the Stargardt's (STGD3) mutation in ELOVL4 causes a central nervous system (CNS) phenotype in humans, including seizures, intellectual disability, spastic quadriplegia, and death.

There have been human cases of children with homozygous inheritance of the STGD3 mutation who make no VLC-SFAs. These children develop extremely dry skin (ichthyosis), seizures, intellectual disability, and spastic quadriplegia, and die within the first few years of life. These patients with recessive mutations in the ELOVL4 gene (Elovl4) also had hypertonic posture, and magnetic resonance imaging (MRI) showed delayed myelination and evidence of cortical atrophy.

The presence of C28 and C30 SFAs, but not VLC-PUFA, has previously been reported in rat brain sphingolipids. Animal models have been generated that express Elovl4 mutations, but the role of VLC-FAs and the mechanism behind neurodegeneration due to mutations in this gene could not be determined due to neonatal lethality in the mice. Four different studies showed that mice with global deletion or mutation of Elovl4 die within hours after birth from dehydration due to absence of omega-O-acyl-ceramides containing VLC-SFAs in their skin, which provide the epidermal water barrier. Therefore, a skin-expressing double mutant knockin that only expresses functional ELOVL4 in the skin has been developed as part of the present disclosure. This “skin-rescued” mouse doesn't have the skin phenotype, thus allowing the effect on brains of mice with no functional ELOVL4 in the brain to be examined.

### BRIEF DESCRIPTION OF THE DRAWINGS

FIG. 1 is a photograph of a normal wild-type mouse (top) and a “skin rescued” double STGD3 knock-in mouse undergoing a seizure at about 19 days after birth (bottom).

FIG. 2 shows radioactive fluoro-deoxyglucose uptake in various tissues in ELOVL4/ELOVL4 wild-type mice and “skin rescued” STGD3/STGD3 knock-in mice.

FIG. 3 shows GC-MS spectrographs of SFAs extracted from beeswax (upper panel) then enriched by selective precipitation in methanol (lower panel).

FIG. 4 is a graph showing uptake in mice of orally-fed VLC-SFA (extracted from beeswax) in safflower oil (“SFO+bwax”) as pmoles/100 μL plasma (right bars), using standard SFAs such as 25:0 as internal standards. Control (“SFO”) was safflower oil in mouse feed pellets (left bars).

### DETAILED DESCRIPTION

The present disclosure is directed, in at least certain embodiments, to nutritional supplements containing VLC-SFAs for treatment or inhibition of diseases resulting from or caused by mutations in the Elovl4 gene which encodes the ELOVL4 enzyme. In at least certain embodiments, the VLC-SFAs are conjugated to a carrier molecule to form a VLC-SFA carrier conjugate. The carrier may be glycerol, whereby the VLC-SFA carrier conjugate is a VLC-SFA monoglyceride. The present disclosure is further directed, in at least certain embodiments, to methods of using VLC-SFA compositions such as nutritional supplements or vitamin supplements for treatment or inhibition of diseases or conditions involving insufficient production of endogenous VLC-SFAs caused by deficient ELOVL4 enzyme. For example, supplementation of VLC-SFAs can result in incorporation of VLC-SFAs into target tissues such as (but not limited to) brain, skin, retina, and testes.

Before further describing various embodiments of the compounds, compositions, and methods of the present disclosure in more detail by way of exemplary description, examples, and results, it is to be understood that the compounds, compositions, and methods of the present disclosure are not limited in application to the details of specific embodiments and examples as set forth in the following description. The description provided herein is intended for purposes of illustration only and is not intended to be construed in a limiting sense. As such, the language used herein is intended to be given the broadest possible scope and meaning, and the embodiments and examples are meant to be exemplary, not exhaustive. Also, it is to be understood that the phraseology and terminology employed herein is for the purpose of description only and should not be regarded as limiting unless otherwise indicated as so. Moreover, in the following detailed description, numerous specific details are set forth in order to provide a more thorough understanding of the present disclosure. However, it will be apparent to a person having ordinary skill in the art that the present disclosure may be practiced without these specific details. In other instances, features which are well known to persons of ordinary skill in the art have not been described in detail to avoid unnecessary complication of the description. It is intended that all alternatives, substitutions, modifications, and equivalents apparent to those having ordinary skill in the art are included within the scope of the present disclosure. All of the compounds, compositions, and methods of production and application and use thereof disclosed herein can be made and executed without undue experimentation in light of the present disclosure. Thus, while the compounds, compositions, and methods of the present disclosure have been described in terms of particular embodiments, it will be apparent to those of skill in the art that variations may be applied to the compounds, compositions, and/or methods and in the steps or in the sequence of steps of the methods described herein without departing from the concept, spirit, and scope of the inventive concepts.

All patents, published patent applications, and non-patent publications mentioned in the specification or referenced in any portion of this application are herein expressly incorporated by reference in their entirety to the same extent as if each individual patent or publication was specifically and individually indicated to be incorporated by reference.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

As utilized in accordance with the methods and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, includes ranges of 1-20, 10-50, 50-100, 100-500, and 500-1,000, for example.

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the term “about” is used to indicate that a value includes the inherent variation of error for the composition, the method used to administer the composition, or the variation that exists among the study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The term “about” or “approximately”, where used herein when referring to a measurable value such as an amount, a temporal duration, and the like, is meant to encompass, for example, variations of +10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein, any reference to “one embodiment” or “an embodiment” means that a particular element, feature, structure, or characteristic described in connection with the embodiment is included in at least one embodiment and may be included in other embodiments. The appearances of the phrase “in one embodiment” in various places in the specification are not necessarily all referring to the same embodiment and are not necessarily limited to a single or particular embodiment.

The term “pharmaceutically acceptable” refers to compounds and compositions which are suitable for administration to humans and/or animals without undue adverse side effects such as toxicity, irritation, and/or allergic response commensurate with a reasonable benefit/risk ratio. The compounds (e.g., VLC-SFAs) of the present disclosure may be combined with one or more pharmaceutically-acceptable excipients, including carriers, vehicles, and diluents which may improve solubility, deliverability, dispersion, stability, and/or conformational integrity of the compounds or conjugates thereof.

As used herein, “pure,” or “substantially pure” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

Non-limiting examples of animals within the scope and meaning of this term include dogs, cats, rats, mice, guinea pigs, chinchillas, horses, goats, cattle, sheep, zoo animals, Old and New World monkeys, non-human primates, and humans.

“Treatment” refers to therapeutic treatments. “Prevention” refers to prophylactic or preventative treatment measures or reducing the onset of a condition or disease. The term “treating” refers to administering the composition to a subject for therapeutic purposes and/or for prevention. Non-limiting examples of modes of administration include oral, topical, retrobulbar, subconjunctival, transdermal, parenteral, subcutaneous, intranasal, intramuscular, intraperitoneal, intravitreal, and intravenous routes, including both local and systemic applications. The term “topical” is used herein to define a mode of administration through an epithelial surface, such as but not limited to, the skin, eye, or internal epithelial surfaces. In addition, the compositions of the present disclosure may be designed to provide delayed, controlled, extended, and/or sustained release using formulation techniques which are well known in the art.

The terms “therapeutic composition” and “pharmaceutical composition” refer to a composition containing a VLC-SFA as described herein that may be administered to a subject by any method known in the art or otherwise contemplated herein, wherein administration of the composition brings about a therapeutic effect as described elsewhere herein.

The term “effective amount” refers to an amount of a free VLC-SFA or VLA-SFA conjugate which is sufficient to exhibit a detectable therapeutic, amelioration, or treatment effect in a subject without excessive adverse side effects (such as substantial toxicity, irritation and allergic response) commensurate with a reasonable benefit/risk ratio when used in the manner of the present disclosure. The effective amount for a subject will depend upon the subject's type, size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

The term “ameliorate” means a detectable or measurable improvement in a subject's condition or symptom thereof. A detectable or measurable improvement includes a subjective or objective decrease, reduction, inhibition, suppression, limit, or control in the occurrence, frequency, severity, progression, or duration of the condition, or an improvement in a symptom or an underlying cause or a consequence of the condition, or a reversal of the condition. A successful treatment outcome can lead to a “therapeutic effect,” or “benefit” of ameliorating, decreasing, reducing, inhibiting, suppressing, limiting, controlling, or preventing the occurrence, frequency, severity, progression, or duration of a condition, or consequences of the condition in a subject.

A decrease or reduction in worsening, such as stabilizing the condition, is also a successful treatment outcome. A therapeutic benefit therefore need not be complete ablation or reversal of the condition, or any one, most or all adverse symptoms, complications, consequences or underlying causes associated with the condition. Thus, a satisfactory endpoint may be achieved when there is an incremental improvement such as a partial decrease, reduction, inhibition, suppression, limit, control or prevention in the occurrence, frequency, severity, progression, or duration, or inhibition or reversal of the condition (e.g., stabilizing), over a short or long duration of time (e.g., seconds, minutes, hours).

As noted above, mice with global deletion or mutation of Elovl4 die within hours after birth from dehydration due to absence of omega-O-acyl-ceramides containing VLC-SFAs in their skin. The skin phenotype in these mice has been rescued by overexpressing ELOVL4 only in the skin under the control of two different skin-specific promoters (discussed below). These animals survive for up to 21 days. Surprisingly, starting at around day 19, these mice develop a phenotype (running seizures and hyperactivity) that mimics that reported for humans with bi-allelic mutations in ELOVL4. Thus, an animal model has been identified that recapitulates the human condition, which to the inventors' knowledge is the first of its kind and significantly enhances the understanding of the role of VLC-SFAs in brain development and neuronal function. This is the first report that causally links mutation in ELOVL4 to a neurodegenerative disease.

As described herein, it has been discovered that ELOVL4-synthesized VLC-SFAs play an essential role in neural cell structure and function including but not limited to the maintenance and regulation of neural signaling in the brain. The role of ELOVL4 and VLC-FAs in the brain has been an otherwise unstudied area of research. Results have been generated that indicate that ELOVL4 and VLC-SFAs are involved in a severe neurodegenerative phenotype. A unique mouse line has been generated that recapitulates the human neurological phenotype, which until recently, was unknown and not amenable to investigation. These unique animal models have allowed the mechanism of this disease to be probed. Although VLC-SFAs are difficult to study due to their solubility properties and low levels in tissues, the present disclosure is the first to demonstrate the enzymatic function of ELOVL4.

As noted above, the present disclosure is directed, in certain embodiments, to nutritional supplements containing VLC-SFAs for treatment or inhibition of diseases resulting from or caused by Elovl4 mutations. Incorporation of VLC-SFAs into target tissues (brain, skin, retina, and testes) can be via oral, intravenous, or parenteral supplementation for example, using these nutritional supplements.

Thus, the compositions of the present disclosure include, in various non-limiting embodiments, a dietary supplement, a vitamin supplement (such as, but not limited to, a prenatal vitamin supplement), a food product, a nutritional formulation, a pharmaceutical formulation, a humanized animal milk, an infant formula, and a cosmetic item, each containing VLC-SFAs as disclosed herein. The presently disclosed VLC-SFA formulations can be used as a treatment for at least one of, but not limited to: (1) enhancing neural development and function, (2) a neurodegenerative disease, (3) an ocular disorder, (4) a retinal disorder, (5) a skin disorder, and (6) any disease resulting from mutations in ELOVL4.

More particularly, the VLC-SFAs referred to herein may have a carbon chain length of at least about 28 carbons (such as, but not limited to, at least 28 carbons, at least 29 carbons, at least 30 carbons, at least 31 carbons, at least 32 carbons, at least 33 carbons, at least 34 carbons, at least 35 carbons, at least 36 carbons, at least 37 carbons, at least 38 carbons, at least 39 carbons, or at least 40 carbons). The VLC-SFAs of the present disclosure can be obtained by purification from natural lipid sources such as beeswax or by synthetic production via the ELOVL4 elongase enzyme (e.g., using synthesis methods as described in U.S. Pat. No. 8,021,874), for example using C24 and/or C26 SFA precursor molecules.

The nutritional compositions (supplements) of the present disclosure can be administered to a mammalian subject orally, parenterally, intravenously, or otherwise internally. One nutritional composition for internal consumption is an infant formula or an oral dosage form for treatment or prevention of a disease condition, or a vitamin supplement which includes one or more VLC-SFAs or VLC-SFA conjugates as described herein. In one embodiment, the nutritional compositions are in a liquid form or a solid form as discussed elsewhere herein. In one non-limiting embodiment of a dietary supplement, the VLC-SFAs are dissolved in a vegetable oil and supplied, for example, at a rate of from about 1 mg to about 1000 mg of VLC-SFA per day, from about 10 mg to about 750 mg of VLC-SFA per day, from about 50 mg to about 500 mg VLC-SFA per day, or any suitable rate determined for a particular subject or patient. For commercial sale for research or other purposes, specific VLC-SFAs can be supplied as a substantially pure substance (e.g., about 99% pure) sealed under an inert gas such as nitrogen or argon and sold in amounts, for example, ranging from about 1 mg to about 1 gm, as free fatty acids, free fatty acid salts, or in whole lipid form.

The VLC-SFAs of the compositions or supplements of the present disclosure may also be provided as a carrier molecule conjugate wherein the VLC-SFA is linked or otherwise conjugated to a carrier molecule such as a single VLC-SFA molecule conjugated to a glycerol molecule, e.g., as a VLC-SFA monoglyceride. Such VLC-SFA monoglycerides can be synthesized using standard methods known to those having ordinary skill in the art. For example, free VLC-SFAs can be reacted with thionyl chloride to form VLC-SFA chlorides (Organic Syntheses, Coll. Vol. 1, p. 147 (1941); Vol. 9, p. 32 (1929)), which are then combined with excess glycerol resulting in the production of primarily VLC-SFA monoglycerides via an exothermic reaction. The VLC-SFA monoglycerides can also be synthesized using commercially-available lipases (e.g., Fregolente et al., Appl. Biochem Biotechnol (2008) 146:165-172). Other non-limiting examples of carrier molecules to which VLC-SFAs disclosed herein may be conjugated include polyols such as, but not limited to, erythritol, isomalt, lactitol, maltitol, mannitol, sorbitol, xylitol, and inositol.

In some embodiments, infant formulas of the present disclosure typically contain proteins, carbohydrates, lipids, vitamins, minerals, and other nutrients, and are commercially available as reconstitutable powders, ready-to-feed liquids, and dilutable liquid concentrates. As such, certain embodiments disclosed herein are directed to methods of providing nutrition to an infant, toddler, child, or adult, said methods comprising the administration or feeding of a nutritional composition of the present disclosure to the infant, toddler, child, or adult as a nutritional formula or supplement as their sole, primary, or partial nutritional needs.

The compositions of the present disclosure may comprise protein, carbohydrate, and lipids, as well as the one or more VLC-SFAs (free or conjugated) described herein above, and may further contain combinations of antioxidant carotenoids including (but not limited to) lutein, lycopene, and beta-carotene. These and other elements of the nutritional formulas and corresponding methods are described elsewhere herein and are known in the art. The compositions may further comprise vitamins, minerals, and electrolytes, and may potentially serve as the sole source of nutrition when provided in sufficient quantity.

In some embodiments, the present disclosure is directed to methods of treating a patient or subject to mitigate a disease or condition such as, but not limited to, Stargardt disease, cortical atrophy, delayed myelinization, seizure disorders, mental retardation, erythematous ichthyosis, retinal diseases, or any disease or condition caused by a mutation in ELOVL4 (or treating subjects or patients to prevent or delay progression of such diseases) by administration of the compositions described or otherwise contemplated herein, for example (but not by way of limitation) either by oral supplementation, dietary supplementation, or intravenous supplementation such as via a PICC line, or any other suitable means of delivery. Stargardt-like macular dystrophies are a group of progressive photoreceptor degenerative disorders that eventually lead to loss of vision. Three mutations in the Elovl4 gene are known to cause one form of the disease named Stargardt-3 (STGD3).

Experimental Results

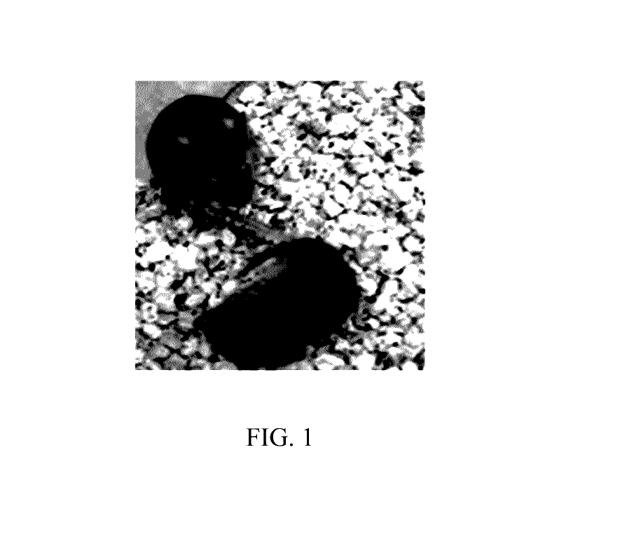

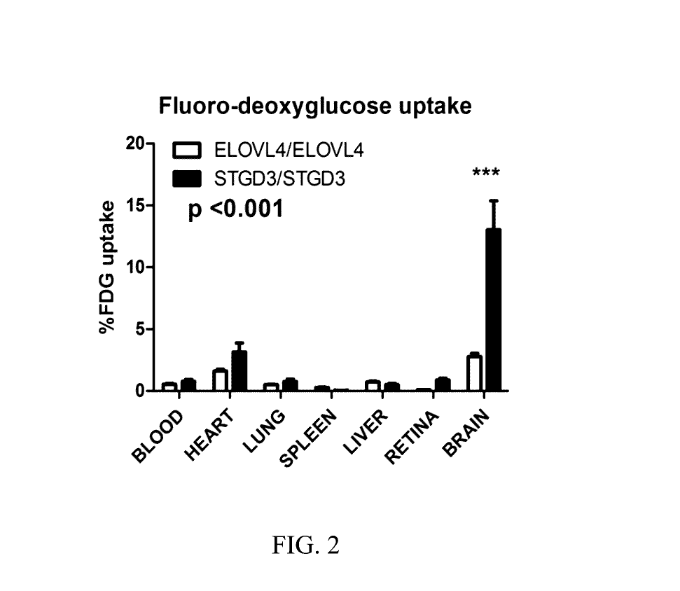

Mice homozygous for the mutant Elovl4 gene (Elovl4 mut/mut) or with global deletion of Elovl4 (Elovl4−/−) die within hours after birth from dehydration due to the absence of omega-O-acyl-ceramides containing VLC-SFAs in their skin, which provide the epidermal water barrier. To circumvent the skin defect, two human skin-specific promoters, keratin-14 (K-14) and involucrin, were used to express the wild-type Elovl4 gene in skin of a double STGD3 knock-in mouse, allowing the impact of the global bi-allelic mutations to be studied while maintaining the integrity of the skin's water permeability barrier. The lethality problem was partially resolved. These skin-rescued knock-in animals survive from birth for approximately 21 days. Around P19, they begin to develop running seizures (FIG. 1), and two days later they die. Upon noting this phenotype, the 19-day-old mice and littermate controls (n=5 each) were exposed to for Positron Emission Tomography (PET) imaging to evaluate the uptake of 18F-fluoro-deoxyglucose, a marker for glucose metabolism in the central nervous system (CNS). The results were fascinating in that they revealed a near 3-fold increase in the amount of glucose taken up into the brains of these seizure-prone animals. This result was confirmed quantitatively by measuring the amount of 18F radioactivity in each tissue, harvested post mortem (FIG. 2). These results indicate that there is an increase in neuronal metabolism, which would correlate with the seizure activity, a disruption in glycolysis, which would increase the cellular demand for glucose, or a breakdown of the blood brain barrier (BBB). The latter (BBB breakdown) was eliminated by doing magnetic resonance imaging (MRI) analysis of gadolinium uptake in mutant and WT mice; no significant difference was found in the relative change in MRI signal intensity (post-contrast minus pre-contrast). Magnetic resonance spectroscopy was performed in these mice, and no gross differences were found in the levels of N-acetylaspartate, choline, and creatine relative to water.

Prior to the present disclosure, the specific pattern of ELOVL4 expression within the mouse brain was unknown, and its CNS products were unidentified. Using immunohistochemistry with an antibody against mouse ELOVL4 protein generated as part of the present disclosure, the regions within the brain that express ELOVL4 were identified. ELOVL4 staining was detected in the cortex, cerebellum, and olfactory bulbs, but the highest expression was in the medial temporal lobe within neurons (not glia) in the sub-granular layer of the dentate gyrus in the hippocampus.

It has previously been reported that sphingolipids from whole rat brain analyzed by gas chromatography-mass spectrometry (GC-MS) contain VLC-SFAs, but no very long chain polyunsaturated fatty acids (VLC-PUFAs). The brain total VLC-SFAs in sphingolipids were second only to those in the skin in relative abundance. More recently, both GC-MS and triple quadrupole tandem MS were used to analyze various brain regions (cortex, cerebellum, whole hippocampus, and micro-dissected CA1, CA3, or DG regions from hippocampus) for VLC-PUFA, but again these polyunsaturated fatty acids could not be detected in any region. However, all regions contained VLC-SFA.

Therefore, a feeding study has been completed that shows that VLC-SFA supplemented in the feed of mice for one week are absorbed in the gut and are detected in the plasma (shown below). This demonstrates the effectiveness of using VLC-SFAs as an oral supplement. Also, zebrafish given morpholinos to knock down ELOVL4 and supplemented with VLC-SFAs in their egg water developed with normal skin and did not die as opposed to those not supplemented with VLC-SFA in their egg water.

Feeding Study:

Preparation and Successful Uptake of Very Long-Chain Saturated Fatty Acids (VLC-SFA) Prepared from Wax Esters in Beeswax

As noted above, the elongase ELOVL4 is necessary for the synthesis of VLC-FAs (very long chain fatty acids, ≧C28). ELOVL4 is necessary for the synthesis of both VLC-SFAs and VLC-PUFAs (Agbaga M. et al., Role of Stargardt-3 macular dystrophy protein (ELOVL4) in the biosynthesis of very long chain fatty acids, PNAS (2008) 105(35):12843-12848). VLC-SFAs are found in the skin and brain. Mutations in ELOVL4 lead to compromised skin permeability barrier function, retinal degenerations, and improper brain development and function (Vasireddy, V. et al. Loss of functional ELOVL4 depletes very long-chain fatty acids and unique acyleramides in skin leading to neonatal death, Hum. Mol. Genet. (2007) 16(5):471-482).

Methods:

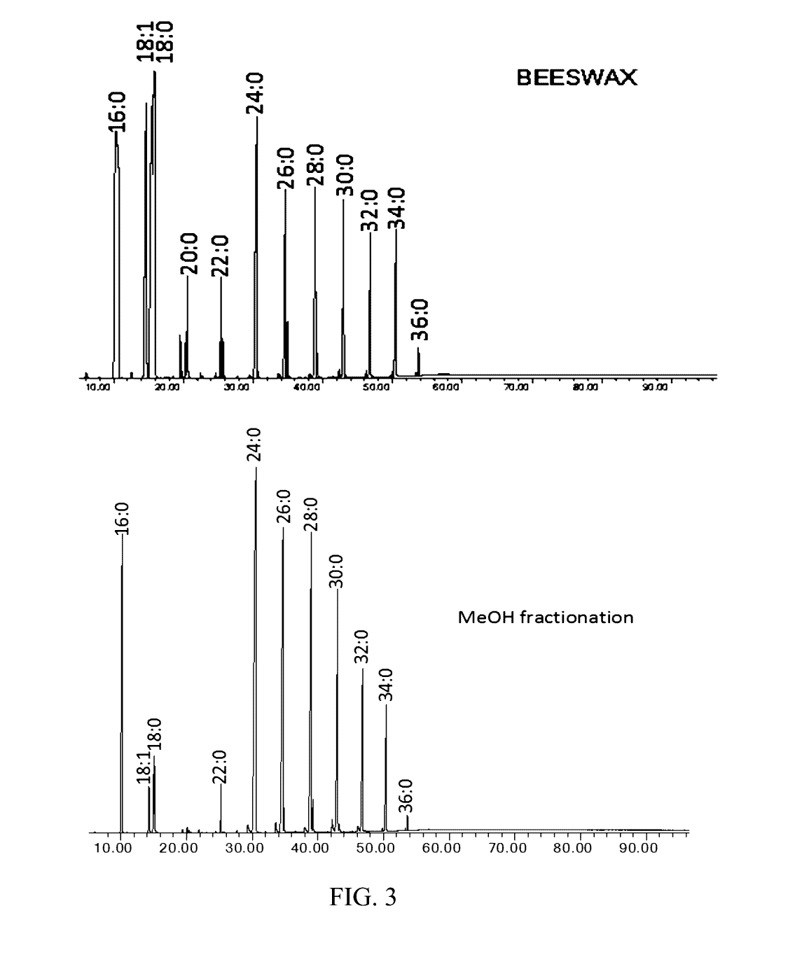

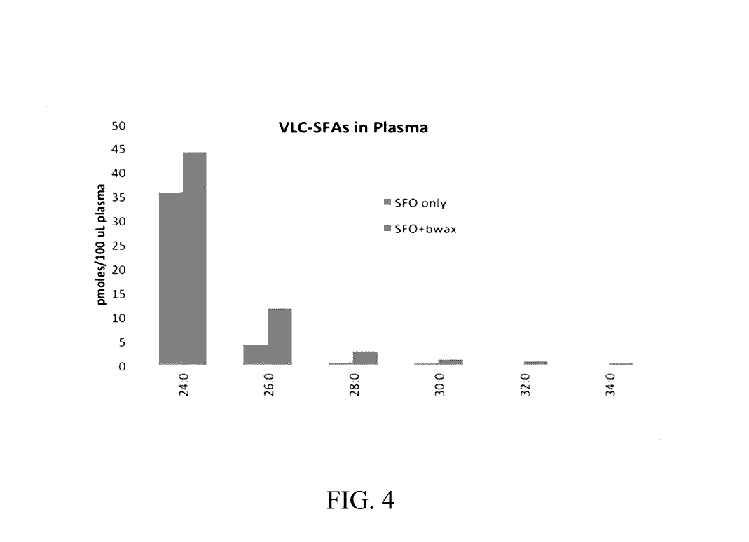

Lipids from pharmaceutical grade yellow beeswax were saponified in 2% KOH in EtOH. Saturated fatty acids (SFAs) were extracted into hexane under acidic conditions. VLC-SFAs were enriched by selective precipitation in methanol. The composition was verified by GC-MS (FIG. 3). The VLC-SFAs were mixed with safflower oil and incorporated into mouse feed. The mice were euthanized after feeding for 4 days, and plasma was collected for analysis. Lipids were isolated from plasma by Chloroform/Methanol (1:1) extraction. Diethylenetriaminepentaacetic acid (DTPA) was added to chelate and remove iron. The resulting organic layer was dried. Standard SFAs (C15/17, C25/27) were added as internal standards. A C25-30 mix standard was used as an external standard. Fatty acid methyl esters (FAMES) were made by heating in 16% HCl in methanol. FAMES were extracted into hexane, dried under nitrogen, and developed on silica gel 60 TLC plates to remove cholesterol. FAMES were recovered from TLC plates and analyzed by GC-MS to determine the presence and amounts of VLC-SFAs. Results were compared to the control set (FIG. 4).

The present disclosure proposed that a lack of VLC-FAs causes detrimental phenotypes. Therefore, VLC-SFAs were isolated from beeswax and incorporated into mouse feed to determine if they could be absorbed. Beeswax lipids were soponified, and VLC-SFAs (very long chain saturated fatty acids, C28-C34) were enriched. The VLC-SFA's composition was verified by Gas Chromatography-Mass Spectrometry (GC-MS), as shown in FIG. 3. The isolated VLC-SFAs were mixed with safflower oil and incorporated into mouse feed. The mice were fed for 4 days then euthanized and plasma was collected. Upon lipid extraction of the plasma and methyl ester derivatization of the fatty acids, fatty acid methyl esters (FAMEs) were analyzed by GC-MS to determine the presence and amounts of VLC-SFAs in the plasma (FIG. 4). The results were compared to a control group given chow with safflower oil (SFO) only. GC-MS analysis showed VLC-SFAs were isolated from beeswax with an average 13.6% yield. GC-MS revealed a significant increase of VLC-SFAs in the plasma of mice given VLC-SFA feed (5.20 pmoles/100 μL plasma) compared to safflower oil controls (1.33 pmoles/100 μL). The trace amount of 28C and 30C SFAs identified in the plasma of the non-supplemented controls may have been due to a basal level of VLC-SFAs therein. These results show that orally-provided VLC-SFAs can be absorbed by the intestine and transported by the blood.

In certain embodiments, the present disclosure includes a method of treating a subject for a condition related to an ELOVL4 deficiency, in which an effective amount of a VLC-SFA composition (such as, but not limited to, a VLC-SFA monoglyceride) is administered to a subject in need of such therapy. An effective amount of a VLC-SFA composition of the present disclosure will generally contain sufficient active substance to deliver from about 0.01 μg/kg to about 100 mg/kg (weight of VLC-SFA/body weight of the subject) to the subject. For example, the composition will deliver from about 0.1 μg/kg to about 50 mg/kg, and more particularly from about 1 μg/kg to about 20 mg/kg, to the subject.

Practice of the methods of the present disclosure may comprise, in at least one embodiment, administration of an effective amount of the VLC-SFA to a subject in any suitable systemic and/or local formulation, in an amount effective to deliver a dosage disclosed herein. The dosage can be administered, for example but not by way of limitation, on a one-time basis, or administered at multiple times (for example, but not by way of limitation, from one to five times per day, or once or twice per week), or continuously via a venous drip, depending on the desired therapeutic effect. In one non-limiting example of a therapeutic method of the present disclosure, the dosage is provided in an IV infusion in the range of from about 0.01 mg/kg to about 10 mg/kg of body weight once a day.

Administration of the free or conjugated VLC-SFA used in the pharmaceutical composition or to practice the method of the present disclosure can be carried out in a variety of conventional ways, such as, but not limited to, orally, by inhalation, rectally, topically, nasally, or by cutaneous, subcutaneous, intraperitoneal, vaginal, or intravenous injection. Oral formulations may be formulated such that the VLC-SFA passes through a portion of the digestive system before being released; for example, it may not be released until reaching the small intestine.

When an effective amount of the VLC-SFA composition is administered orally, it may be in the form of a solid or liquid preparation, such as (but not limited to) a capsule, a pill, a tablet, a lozenge, a melt, a powder, a suspension, a solution, an elixir, an emulsion, or any combination thereof. Solid unit dosage forms can be capsules of the ordinary gelatin type containing, for example, surfactants, lubricants, and inert fillers such as lactose, sucrose, and cornstarch; alternatively (and/or in addition thereto), the dosage forms can be sustained release preparations. The pharmaceutical composition may contain a solid carrier, such as (but not limited to) a gelatin or an adjuvant. The tablet, capsule, and powder may contain from about 0.05 to about 95% of the active substance compound by dry weight. When administered in liquid form, a liquid carrier such as (but not limited to) water, petroleum, one or more oils of animal or plant origin (such as, but not limited to, peanut oil, mineral oil, soybean oil, or sesame oil), or one or more synthetic oils may be added. The liquid form of the pharmaceutical composition may further contain physiological saline solution, dextrose or other saccharide solution, or glycols such as ethylene glycol, propylene glycol, or polyethylene glycol. When administered in liquid form, the pharmaceutical composition particularly contains from about 0.005 to about 95% by weight of the active substance. For example, a dose of from about 10 mg to about 1000 mg once or twice a day could be administered orally. As noted above, in at least one embodiment, the composition comprises a VLC-SFA monoglyceride conjugate.

In another embodiment, the VLC-SFAs of the present disclosure can be tableted with conventional tablet bases (such as, but not limited to, lactose, sucrose, and cornstarch) in combination with binders (such as, but not limited to, acacia, cornstarch, or gelatin), disintegrating agents (such as, but not limited to, potato starch or alginic acid), and a lubricant (such as, but not limited to, stearic acid or magnesium stearate). Liquid preparations are prepared by dissolving the VLC-SFAs in an aqueous or non-aqueous pharmaceutically acceptable solvent which may also contain suspending agents, sweetening agents, flavoring agents, and preservative agents as are known in the art.

For parenteral administration, for example, the VLC-SFA compositions may be dissolved in a physiologically acceptable pharmaceutical carrier and administered as either a solution or a suspension. Illustrative of suitable pharmaceutical carriers are water, saline, dextrose solutions, fructose solutions, ethanol, or oils of animal, vegetative, or synthetic origin. The pharmaceutical carrier may also contain preservatives and buffers as are known in the art.

When an effective amount of the VLC-SFA composition is administered by intravenous, cutaneous, or subcutaneous injection, it may be in the form of a pyrogen-free, parenterally acceptable aqueous solution or suspension. The preparation of such parenterally acceptable solutions, having due regard to pH, isotonicity, stability, and the like, is well within the skill in the art. A particular pharmaceutical composition for intravenous, cutaneous, or subcutaneous injection may contain, in addition to the peptide compound, an isotonic vehicle such as Sodium Chloride Injection, Ringer's Injection, Dextrose Injection, Dextrose and Sodium Chloride Injection, Lactated Ringer's Injection, or other vehicle as known in the art. The compositions of the present disclosure may also contain stabilizers, preservatives, buffers, antioxidants, or other additives known to those of skill in the art.

As noted, particular amounts and modes of administration can be determined by one of ordinary skill in the art. One of ordinary skill in the art of preparing formulations can readily select the proper form and mode of administration, depending upon the particular characteristics of the VLC-SFA compositions selected, the condition to be treated, the stage of the condition, and other relevant circumstances using formulation technology known in the art, described, for example, in Remington: The Science and Practice of Pharmacy, 21^{st} ed. (Philadelphia, Pa. Lippincott Williams & Wilkins. 2005).

Additional pharmaceutical methods may be employed to control the duration of action of the VLC-SFA compositions. Increased half-life and/or controlled release preparations may be achieved through the use of proteins or polymers to conjugate, complex with, and/or absorb the VLC-SFAs as discussed previously herein. The controlled delivery and/or increased half-life may be achieved by selecting appropriate macromolecules (for example but not by way of limitation, polysaccharides, polyesters, polyamino acids, homopolymers, polyvinyl pyrrolidone, ethylenevinylacetate, methylcellulose, or carboxymethylcellulose, and acrylamides such as N-(2-hydroxypropyl) methacrylamide), and the appropriate concentration of macromolecules as well as the methods of incorporation, in order to control release.

Another possible method useful in controlling the duration of action of the VLC-SFA composition by controlled release preparations and half-life is incorporation of the VLC-SFA composition into particles of a polymeric material such as polyesters, polyamides, polyamino acids, hydrogels, poly(lactic acid), ethylene vinylacetate copolymers, copolymer micelles of, for example, polyethylene glycol (PEG) and poly(l-aspartamide).

It is also possible to entrap the VLC-SFA composition in microcapsules prepared, for example, by coacervation techniques or by interfacial polymerization (for example, hydroxymethylcellulose or gelatine-microcapsules and poly-(methylmethacylate) microcapsules, respectively), in colloidal drug delivery systems (for example, liposomes, albumin microspheres, microemulsions, nano-particles, and nanocapsules), or in macroemulsions. Such techniques are well known to persons having ordinary skill in the art.

When the VLC-SFA composition is to be used as an injectable material, it can be formulated into a conventional injectable carrier. Suitable carriers include (but are not limited to) biocompatible and pharmaceutically acceptable phosphate buffered saline solutions, which are particularly isotonic.

For reconstitution of a lyophilized product in accordance with the present disclosure, one may employ a sterile diluent, which may contain materials generally recognized for approximating physiological conditions and/or as required by governmental regulation. In this respect, the sterile diluent may contain a buffering agent to obtain a physiologically acceptable pH, such as (but not limited to) sodium chloride, saline, phosphate-buffered saline, and/or other substances which are physiologically acceptable and/or safe for use. In general, the material for intravenous injection in humans should conform to regulations established by the US Food and Drug Administration, which are available to those in the field. The pharmaceutical composition may also be in the form of an aqueous solution containing many of the same substances as described above for the reconstitution of a lyophilized product.

The compounds, conjugates, compositions, and methods of production and application of the VLC-SFAs described herein can be made and executed without undue experimentation in light of the present disclosure. While the present disclosure has been described in connection with certain embodiments so that aspects thereof may be more fully understood and appreciated, it is not intended that the present disclosure be limited to these particular embodiments. On the contrary, it is intended that all alternatives, modifications, and equivalents are included within the scope of the present disclosure. Thus the examples described above, which include particular embodiments, will serve to illustrate the practice of the present disclosure, it being understood that the particulars shown are by way of example and for purposes of illustrative discussion of particular embodiments only and are presented in the cause of providing what is believed to be the most useful and readily understood description of procedures as well as of the principles and conceptual aspects of the presently disclosed methods and compositions. Changes may be made in the formulation of the various compositions described herein, the methods described herein or in the steps or the sequence of steps of the methods described herein without departing from the spirit and scope of the present disclosure.

What is claimed is:

1. A fatty acid composition, comprising:
at least one purified very long chain saturated fatty acid (VLC-SFA) monoglyceride comprising a VLC-SFA conjugated to a glycerol molecule, wherein the VLC-SFA has a chain length in a range of from 28 carbons to 40 carbons.

2. A nutritional supplement, comprising:
at least one purified very long chain saturated fatty acid (VLC-SFA) monoglyceride comprising a VLC-SFA conjugated to a glycerol molecule, wherein the VLC-SFA has a chain length in a range of from 28 carbons to 40 carbons.

3. The nutritional supplement of claim 2, further defined as being selected from the group consisting of a dietary supplement, a vitamin supplement, a food product, a nutritional formulation, a pharmaceutical formulation, humanized animal milk, and an infant formula.

4. A method of treating a condition caused by a mutation in the Elongation of Very Long chain fatty acids-4 (ELOVL4) elongase in a subject in need of such therapy, the method comprising:
administering to the subject an effective amount of a fatty acid composition comprising at least one purified very long chain saturated fatty acid (VLC-SFA) monoglyceride comprising a VLC-SFA conjugated to a glycerol molecule, wherein the VLC-SFA has a chain length in a range of from 28 carbons to 40 carbons.
